(19) United States
(12) Patent Application Publication
(10) Pub. No.: US 2015/0337003 A1
(43) Pub. Date: Nov. 26, 2015

(54) ABIRATERONE AND ANALOGS THEREOF FOR THE TREATMENT OF DISEASES ASSOCIATED WITH CORTISOL OVERPRODUCTION

(71) Applicant: CORTENDO AB (PUBL), Radnor, PA (US)

(72) Inventors: Theodore Richard KOZIOL, Wynnewood, PA (US); Joseph H. REISER, Norristown, PA (US)

(21) Appl. No.: 14/760,057

(22) PCT Filed: Jan. 18, 2014
(86) PCT No.: PCT/IB2014/000619

§ 371 Date: Jul. 9, 2015

Related U.S. Application Data
(60) Provisional application No. 61/754,460, filed on Jan. 18, 2013.

(51) Int. Cl. C07J 43/00

(52) U.S. Cl. CPC C07J 43/003

(57) ABSTRACT

Novel analogs of abiraterone and methods of treating diseases associated with the overproduction of cortisol, such as Cushing's syndrome, obesity, headache depression, hypertension, diabetes mellitus type II, metabolic syndrome, pseudo-Cushing syndrome, cognitive impairment, dementia, heart failure, renal failure, psoriasis, glaucoma, cardiovascular disease, stroke or incidentalomas, by administering an effective amount of abiraterone or analogs thereof to a patient in need thereof are described.

### BRIEF SUMMARY

Embodiments described herein are directed toward novel compounds of formula (I),

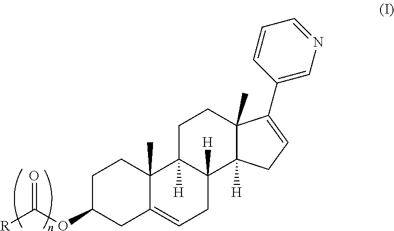

including hydrates, solvates, pharmaceutically acceptable salts, and complexes thereof, wherein:

R is selected from the group consisting of hydrogen, optionally substituted C₁-C₆ linear alkyl, optionally substituted C₃-C₆ branched alkyl, optionally substituted C₃-C₇ cycloalkyl, OR¹, NHR^{2a}, NR^{2a}R^{2b}, and

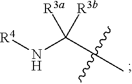

R¹ is selected from the group consisting of optionally substituted C₁-C₆ linear alkyl, optionally substituted C₁-C₆ branched alkyl, and optionally substituted C₃-C₇ cycloalkyl;

R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;

R^{3a} and R^{3b} are each independently selected from a group consisting of hydrogen, optionally substituted C₁-C₆ linear alkyl, optionally substituted C₃-C₆ branched alkyl, optionally substituted benzyl, and optionally substituted heteroarylalkyl:

R⁴ is selected from a group consisting of hydrogen, optionally substituted C₁-C₆ linear alkyl, optionally substituted C₁-C₄ branched alkyl, optionally substituted benzyl, and optionally substituted heteroarylalkyl; and

n is 0 or 1. In some embodiments, the novel compounds of formula (I) do not include abiraterone or abiraterone acetate.

Some embodiments include novel compounds having formula (II):

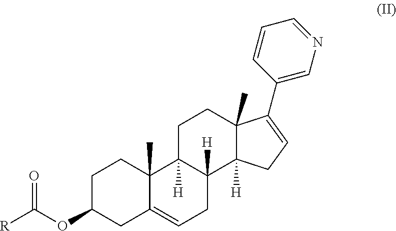

including hydrates, solvates, pharmaceutically acceptable salts, and complexes thereof, wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and

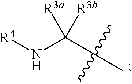

R¹ is selected from the group consisting of optionally substituted C₁-C₆ linear alkyl, optionally substituted C₁-C₆ branched alkyl, and optionally substituted C₃-C₇ cycloalkyl;

R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;

R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl; and

R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl. In some embodiments, the novel compounds of formula (II) does not include abiraterone or abiraterone acetate.

Some embodiments include novel compounds having formula (III):

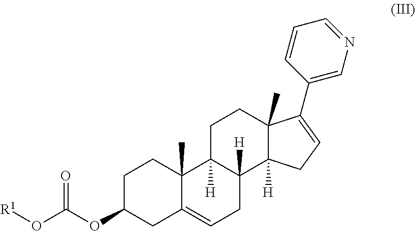

including hydrates, solvates, pharmaceutically acceptable salts, and complexes thereof, wherein R¹ is selected from the group consisting of optionally substituted C₁-C₆ linear alkyl, optionally substituted C₁-C₆ branched alkyl, and optionally substituted C₃-C₇ cycloalkyl.

Some embodiments relate to novel compounds having formula (IV):

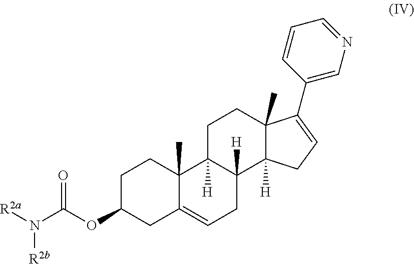

including hydrates, solvates, pharmaceutically acceptable salts, and complexes thereof, wherein R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl.

Some embodiments include novel compounds having formula (V):

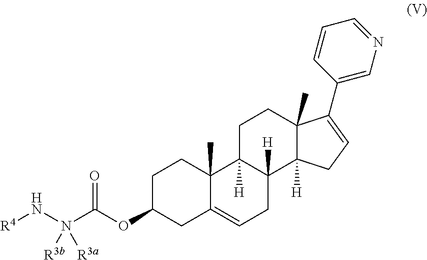

including hydrates, solvates, pharmaceutically acceptable salts, and complexes thereof, wherein R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl; and

R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl.

Some embodiments relate to a composition comprising one or more compounds according to an embodiment herein and an excipient. In some embodiments, the one or more compounds is in an effective amount.

Some embodiments relate to a method for treating, delaying, slowing, or inhibiting the progression of diseases that involve overproduction of cortisol, including, for example, Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, said method comprising administering to a subject in need thereof an effective amount of a compound or composition according to an embodiment herein, wherein the disease that involves overproduction of cortisol is treated, delayed, slowed, or inhibited.

Some embodiments relate to a method for treating, delaying, slowing, or inhibiting the progression of diseases that involve overproduction of cortisol, including, for example, Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, wherein said method comprises administering to a subject a composition comprising an effective amount of one or more compounds according to an embodiment herein and an excipient.

Some embodiments relate to a method for treating, delaying, slowing, or inhibiting the progression of diseases or conditions associated with Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, and diseases that involve overproduction of cortisol, the method comprising administering to a subject an effective amount of a compound or composition according to an embodiment herein.

Some embodiments relate to a method for treating, delaying, slowing, or inhibiting the progression of disease or conditions associated with Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas and diseases that involve overproduction of cortisol, wherein said method comprises administering to a subject a composition comprising an effective amount of one or more compounds according to an embodiment herein and an excipient.

Some embodiments relate to a method for treating, delaying, slowing, or inhibiting the progression of disease or conditions associated with overproduction of cortisol. Said methods comprise administering to a subject an effective amount of a compound or composition according to an embodiment herein.

Some embodiments relate to a method for treating, delaying, slowing, or inhibiting the progression of disease or conditions associated with overproduction of cortisol, wherein said method comprises administering to a subject a composition comprising an effective amount of one or more compounds according to an embodiment herein and an excipient.

Some embodiments relate to a method of lowering the concentration of cortisol in the circulatory system, wherein the method comprises administering to a subject an effective amount of a compound or composition according to an embodiment herein.

Some embodiments relate to a method of lowering the concentration of cortisol in the circulatory system, wherein said method comprises administering to a subject a composition comprising an effective amount of one or more compounds according to an embodiment herein and an excipient.

Some embodiments relate to a process for preparing the compounds of embodiments herein.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present disclosure.

### DETAILED DESCRIPTION

Embodiments herein describe certain novel abiraterone analogs. Embodiments herein also describe methods for using abiraterone and analogs thereof for the treatment of diseases associated with the overproduction of cortisol, such as Cushing's Syndrome. Embodiments herein further describes the use of pro-drugs of abiraterone or analogs thereof for the treatment of diseases associated with the overproduction of cortisol, such as Cushing's Syndrome.

Cortisol is a principal human glucocorticoid exhibiting many important physiological functions. It is involved in the regulation of the metabolism of proteins, carbohydrates, and fats; it counteracts insulin, maintains blood pressure and cardiovascular function, and suppresses the immune system's inflammatory response. However, pathological changes in adrenal and the upstream regulating switches may cause an overproduction of cortisol. One disease associated with overproduction of cortisol is Cushing's syndrome (also termed hypercortisolism). In addition to symptoms like central obesity, headache, and depression in patients with hypercortisolism, overproduction of cortisol is associated with hypertension, diabetes mellitus type II, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas.

There is a long felt need for new treatments for diseases and symptoms associated with the overproduction of cortisol, such as Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, that are both disease-modifying and effective in treating patients that are refractory to current treatments. Embodiments herein identify effective treatment for diseases and symptoms associated with the overproduction of cortisol, such as Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas.

The cortisol lowering agents of the embodiments described herein are capable of treating, delaying, slowing, or inhibiting the progression of diseases associated with the overproduction of cortisol, for example Cushing's Syndrome. Without wishing to be limited by theory, it is believed that cortisol lowering agents of embodiments disclosed herein ameliorate, abate, or otherwise cause to be controlled diseases associated with the overproduction of cortisol, for example Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas.

Throughout this description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps.

As used herein, the term “consists of” or “consisting of” means that the method, use of formulation includes only the elements, steps, or ingredients specifically recited in the particular claimed embodiment or claim.

As used herein, the term “consisting essentially of” or “consists essentially of” means that the only active pharmaceutical ingredient in the formulation or method that treats the specified condition (e.g. Cushing's syndrome) is the specifically recited active pharmaceutical ingredient for treating the specified condition in the particular embodiment or claim; that is, the scope of the claim or embodiment is limited to the specified elements or steps and those that do not materially affect the basic and novel characteristic(s) of the particular embodiment.

In this disclosure, where an element or component is said to be included in and/or selected from a list of recited elements or components, it should be understood that the element or component may be any one of the recited elements or components and may be selected from a group consisting of two or more of the recited elements or components.

The use of the singular herein includes the plural (and vice versa) unless specifically stated otherwise, in addition, where the use of the term “about” is before a quantitative value, the present disclosure also include the specific quantitative value itself, unless specifically stated otherwise. As used herein, the term “about” means plus or minus 10% of the numerical value of the number with which it is being used. Therefore, about 50% means in the range of 45%-65%.

Unless otherwise stated, it should be understood that the order of steps or order fir performing certain actions is immaterial so long as the present disclosure remain operable. Moreover, two or more steps or actions may be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 29 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups may be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, unless otherwise noted, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups may be optionally substituted. Nonlimiting examples of Amyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups may be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group may be covalently linked to the defined chemical structure. Cycloalkyl rings may be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]demayyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undemayyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein al hydrogens of an alkyl group have been replaced with halogens (e.g., —CF₃, —CF₂CF₃). Haloalkyl groups may optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings may be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which may be substituted at one or more carbon atoms of the aromatic and/or saturated or partially saturated fines.

The term “arylalkyl” or “aralkyl” refers to the group-alkyl-aryl, where the alkyl and aryl groups are as defined herein. Aralkyl groups may be optionally substituted in embodiments herein. Examples of arylalkyl groups include, for example, benzyl, 1-phenylethyl, 2-phenylethyl, 3-phenylpropyl, 2-phenylpropyl, fluorenylmethyl and the like.

The term “heteroarylalkyl” refers to the group-alkyl-heteroaryl, where the alkyl and heteroaryl groups are as defined herein. Heteroarylalkyl groups may be optionally substituted in embodiments herein.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group may be oxidized. Heterocycle groups may be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl(valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanol, isochromanyl, indolinyl, isoindolinyl, and decahydro-1H-cycloocta[b]pyrrolyl.

The term “heteroaryl,” whether used alone or as part of another group, shall mean one or more rings baying from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group may be oxidized. Heteroaryl groups may be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiophenyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, winch has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring may have carbon atoms and optionally one or more (e.g., 1 to 3) additional heteroatoms independently selected from nitrogen (N), oxygen (O), or sulfur (S). In some embodiments, the ring may be saturated or partially saturated and may be optionally substituted.

For the purposes of this disclosure, fused ring units, as well as spirocyclic rings, bicyclic rings and the like, which comprise a single heteroatom, will be considered to belong to the cyclic family corresponding to the heteroatom containing ring. For example, 1,2,3,4-tetrahydroquinoline having the formula:

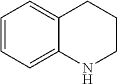

may be, for the purposes of this disclosure, considered a heterocyclic unit. 6,7-Dihydro-5H-cyclopentapyrimidine having the formula:

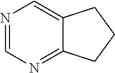

may be, for the purposes of the present disclosure, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

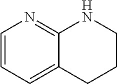

may be, for the purposes of the present disclosure, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

As used herein, the term “substituted” means a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time, in addition, these substituents may replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety may have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, may be optionally substituted. Optionally substituted groups will be so indicated.

The following are non-limiting examples of substituents which may substitute for hydrogen atoms on a moiety: halogen (chlorine (Cl), bromine (Br), fluorine (F) and iodine(l)), —CN, —NO₂, oxo (═O), —OR⁵, —SR⁵, —N(R⁵)₂, —NR⁵C(O)R⁵, —SO₂R⁵, —SO₂OR⁵, —SO₂N(R⁵)₂, —C(O)R⁵, —C(O)OR⁵, —C(O)N(R⁵)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, and R⁵; wherein R⁵, at each occurrence, independently may be hydrogen, —OR⁶, —SR⁶, —C(O)R⁶, —C(O)OR⁶, —C(O)N(R⁶)₂, —SO₂R⁶, —S(O)₂OR⁶, —N(R⁶)₂, —NR⁶C(O)R⁶, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁵ units taken together with the atom(s) to which they are bound may form an optionally substituted carbocycle or heterocycle wherein said carbocycle or heterocycle has 3 to 7 ring atoms; wherein R⁶, at each occurrence, independently may be hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁶ units taken together with the atom(s) to which they are bound may form an optionally substituted carbocycle or heterocycle wherein said carbocycle or heterocycle preferably has 3 to 7 ring atoms.

In some embodiments, the substituents may be selected from:
i) —OR⁷; for example, —OH, —OCH₃, —OCH₂CH₃, —OCH₂CH₂CH;
ii) —C(O)R⁷; for example, —COCH₃, —COCH₂CH₃, —COCH₂CH₂CH₃;
iii) —C(O)OR⁷; for example, —CO₂CH₃, —CO₂CH₂CH₃, —CO₂CH₂CH₂CH₃;
iv) —C(O)N(R⁷)₂; for example, —CONH₂, —CONHCH₃, —CON(CH₃)₂;
v) —N(R⁷)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R⁷; for example, —SO₂H; —SO₂CH₃; —SO₂C₆H₅;
ix) C₁-C₆ linear, branched, or cyclic alkyl;
x) Cyano
xi) Nitro;
xii) N(R⁷)C(O)R⁷;
xiii) Oxo (═O);
xiv) Heterocycle; and
xv) Heteroaryl.
   wherein each R⁷ is independently hydrogen, optionally substituted C₁-C₆ linear or branched alkyl (e.g., optionally substituted C₁-C₄ linear or branched alkyl), or optionally substituted C₃-C₄ cycloalkyl (e.g. optionally substituted C₃-C₄ cycloalkyl); or two R⁷ units may be taken together to form a ring comprising 3-7 ring atoms. In certain aspects, each R⁷ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₅, C₂-C₄, C₂-C₃, C₃-C₆, C₃-C₅, C₃-C₄, C₄-C₆, C₄-C₅, and C₅-C₆, alkyl.

As used herein, the terms “compound,” “analog,” and “composition of matter” stand equally well for the cortisol lowering agent described herein, including all enantiomeric forms, diastereomeric forms, salts, and the like, and the terms “compound,” “analog,” and “composition of matter” are used interchangeably throughout the present specification.

Compounds described herein may contain an asymmetric atom (also referred as a chiral center), and some of the compounds may contain one or more asymmetric atoms or centers, which may thus give rise to optical isomers (enantiomers) and diastereomers. The present disclosure and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers may be obtained in pure form by standard procedures known to those skilled in the art, which include, but are not limited to, diastereomeric salt formation, kinetic resolution, and asymmetric synthesis. The present disclosure also encompass cis and trans isomers of compounds containing alkenyl moieties (e.g., alkenes and imines). It is also understood that the present disclosure encompass all possible regioisomers, and mixtures thereof, which may be obtained in pure form by standard separation procedures known to those skilled in the art, and include, but are not limited to, column chromatography, thin-layer chromatography, and high-performance liquid chromatography.

Pharmaceutically acceptable salts of compounds of the present disclosure, which may have an acidic moiety, may be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or hi-lower alkylamine ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also may be formed. Similarly, when a compound disclosed herein contains a basic moiety, salts may be formed using organic and inorganic acids. For example, salts may be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R⁶)₂, each R⁶ may be the same or different than the other). Combinations of substituents and/or variables may be permissible only if such combinations result in stable compounds.

The terms “treat” and “treating” and “treatment” as used herein, refer to partially or completely alleviating, inhibiting, ameliorating and/or relieving a condition or symptoms thereof from which a patient is suspected to suffer.

A “therapeutically effective amount” or “effective amount” of is a predetermined amount calculated to achieve the desired effect, i.e., to inhibit, or decrease the production of cortisol. The activity contemplated by the present methods includes both medical therapeutic and/or prophylactic treatment, as appropriate. The specific dose of a compound administered according to embodiments described herein to obtain therapeutic and/or prophylactic effects will, of course, be determined by the particular circumstances surrounding the case, including, for example, the compound administered, the route of administration, and the condition being treated. The compounds may be effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of this disclosure in any way. A therapeutically effective amount of compound of embodiments herein is typically an amount such that when it is administered in a physiologically tolerable excipient composition, it is sufficient to achieve an effective systemic concentration or local concentration in the tissue.

Except when noted, the terms “subject” or “patient” may be used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the embodiments described herein may be administered. In an exemplary embodiment, to identify subject patients for treatment according to the embodiments described herein, accepted screening methods may be employed to determine risk factors associated with a targeted or suspected disease or condition or to determine the status of an existing disease or condition in a subject. These screening methods may include, for example, conventional work-ups to determine risk factors that may be associated with the targeted or suspected disease or condition. These and other routine methods allow the clinician to select patients in need of therapy using the methods and compounds of the embodiments described herein.

Some embodiments are directed toward novel compounds of the formula (I),

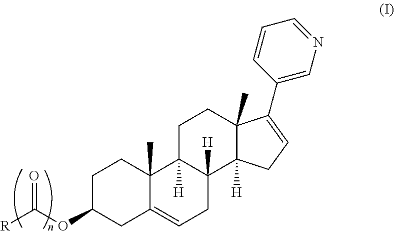

including hydrates, solvates, pharmaceutically acceptable salts, and complexes thereof wherein:

R is selected from the group consisting of hydrogen, optionally substituted C₁-C₆ linear alkyl, optionally substituted C₁-C₄ branched alkyl, optionally substituted C₃-C₇ cycloalkyl, OR¹, NHR^{2a}, NR^{2a}R^{2b}, and

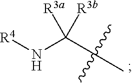

R¹ is selected from the group consisting of optionally substituted C₁-C₆ linear alkyl, optionally substituted C₁-C₆ branched alkyl, and optionally substituted C₃-C₇ cycloalkyl;

R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;

R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;

R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl; and

n is 0 or 1.

Some embodiments include compounds having formula (II):

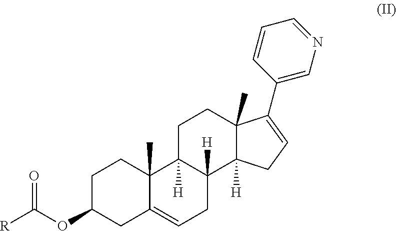

including hydrates, solvates, pharmaceutically acceptable salts, and complexes thereof, wherein:

R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and

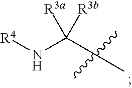

R¹ is selected from the group consisting of optionally substituted C₁-C₆ linear alkyl, optionally substituted C₁-C₆ branched alkyl, and optionally substituted C₃-C₇ cycloalkyl;

R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;

R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl; and

R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl.

In some embodiments, the compounds of formula (I) and formula (II) exclude abiraterone and abiraterone acetate.

Some embodiments include compounds having formula (III):

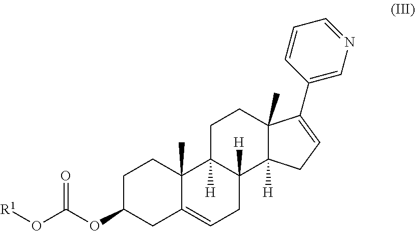

including hydrates, solvates, pharmaceutically acceptable salts, and complexes thereof, wherein R¹ is selected from the group consisting of optionally substituted C₁-C₆ linear alkyl, optionally substituted C₁-C₆ branched alkyl, and optionally substituted C₃-C₇ cycloalkyl.

Some embodiments include compounds having formula (IV):

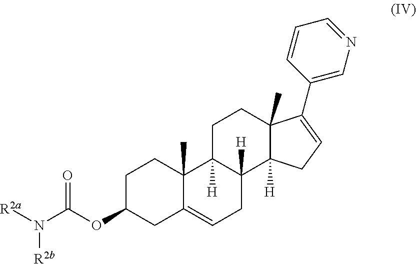

including hydrates, solvates, pharmaceutically acceptable salts, and complexes thereof, wherein R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl.

Some embodiments include compounds having formula (V):

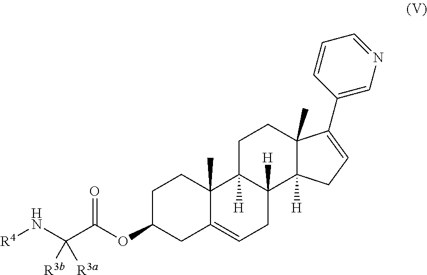

including hydrates, solvates, pharmaceutically acceptable salts, and complexes thereof wherein R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;

R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl;

In some embodiments, R is hydrogen.

In some embodiments, R is optionally substituted C₁-C₆ linear alkyl.

In some embodiments, R is optionally substituted C₁-C₆ branched alkyl.

In some embodiments R is optionally substituted C₃-C₇ cycloalkyl.

In some embodiments R is OR¹.

In some embodiments R is NHR^{2a}

In some embodiments R is NR^{2a}R^{2b}

In some embodiments, R is

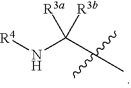

In some embodiments R¹ is optionally substituted C₁-C₆ linear alkyl.

In some embodiments R¹ is optionally substituted C₁-C₆ branched alkyl.

In some embodiments R¹ is optionally substituted C₃-C₇ cycloalkyl.

In some embodiments R^{2a} is hydrogen.

In some embodiments R^{2a} is optionally substituted C₁-C₆ linear alkyl.

In some embodiments R^{2a} is optionally substituted C₁-C₆ branched alkyl.

In some embodiments R^{2a} is optionally substituted C₃-C₇ cycloalkyl.

In some embodiments R^{2b} is hydrogen.

In some embodiments R^{2b} is optionally substituted C₁-C₆ linear alkyl.

In some embodiments R^{2b} is optionally substituted C₁-C₆ branched alkyl.

In some embodiments R^{2b} is optionally substituted C₃-C₇ cycloalkyl.

In some embodiments R^{3a} is hydrogen.

In some embodiments R^{3a} is optionally substituted C₁-C₆ linear alkyl

In some embodiments R^{3a} is optionally substituted C₁-C₆ branched alkyl.

In some embodiments R^{3a} is optionally substituted benzyl.

In some embodiments R^{3b} is optionally substituted heteroarylalkyl.

In some embodiments R^{3b} is hydrogen.

In some embodiments R^{3b} is optionally substituted C₁-C₆ linear alkyl

In some embodiments R^{3b} is optionally substituted C₁-C₆ branched alkyl

In some embodiments, R^{3b} is optionally substituted benzyl.

In some embodiments, R^{3b} is optionally substituted heteroarylalkyl.

In some embodiments, R⁴ is hydrogen.

In some embodiments, R⁴ is optionally substituted C₁-C₆ linear alkyl.

In some embodiments, R⁴ is optionally substituted C₁-C₆ branched alkyl.

In some embodiments, R⁴ is optionally substituted benzyl.

In some embodiments, R⁴ is optionally substituted heteroarylalkyl.

In some embodiments, n is 0.

In some embodiments, n is 1.

Embodiments include a composition comprising a compound selected from the group consisting of (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propionate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyrate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentanoate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexanoate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl heptanoate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isobutyrate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pivalate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dimethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipentylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diisopropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminoacetate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate; (2S,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate; (2R,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate; (2S,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate; (2R,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate; (R)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid; (S)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid; (R)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid; (S)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amine-3-mercaptopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate; and (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate and a pharmaceutically acceptable excipient.

Exemplary embodiments include compounds having the formula (II) or a pharmaceutically acceptable salt form thereof:

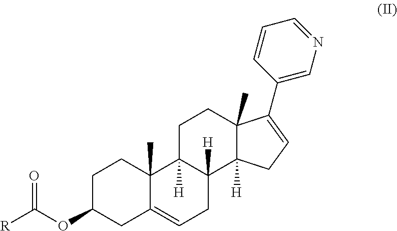

wherein non-limiting examples of R are defined herein below in Table I.

TABLE 1

| Example | R |
|---|---|
| 1 | H |
| 2 | CH₃ |
| 3 | CH₂CH₃ |
| 4 | (CH₂)₂CH₃ |
| 5 | (CH₂)₃CH₃ |
| 6 | (CH₂)₄CH₃ |
| 7 | (CH₂)₅CH₃ |
| 8 | CH(CH₃)₂ |
| 9 | C(CH₃)₃ |

Exemplary embodiments include compounds having the formula (III) or a pharmaceutically acceptable salt form thereof:

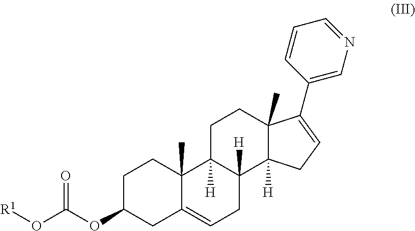

wherein non-limiting examples of R¹ are defined herein below in Table 2.

TABLE 2

| Example | R¹ |
|---|---|
| 10 | —CH₃ |
| 11 | —CH₂CH₃ |
| 12 | —(CH₂)₂CH₃ |
| 13 | —(CH₂)₃CH₃ |
| 14 | —(CH₂)₄CH₃ |
| 15 | —(CH₂)₅CH₃ |
| 16 | —CH(CH₃)₂ |
| 17 | —C(CH₃)₃ |

Exemplary embodiments include compounds having the formula (IV) or a pharmaceutically acceptable salt form thereof:

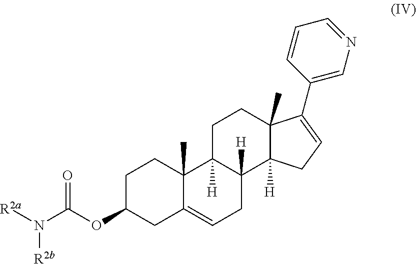

wherein non-limiting examples of R^{2a} and R^{2b} are defined herein below in Table 3.

TABLE 3

| Example | R^{2a} | R^{2b} |
|---|---|---|
| 18 | H | CH₃ |
| 19 | H | CH₂CH₃ |
| 20 | H | (CH₂)₂CH₃ |
| 21 | H | (CH₂)₃CH₃ |
| 22 | H | (CH₂)₄CH₃ |
| 23 | H | (CH₂)₅CH₃ |
| 24 | H | CH(CH₃)₂ |
| 25 | H | C(CH₃)₃ |
| 26 | CH₃ | CH₃ |
| 27 | CH₂CH₃ | CH₂CH₃ |
| 28 | (CH₂)₂CH₃ | (CH₂)₂CH₃ |
| 29 | (CH₂)₃CH₃ | (CH₂)₃CH₃ |
| 30 | (CH₂)₄CH₃ | (CH₂)₄CH₃ |
| 31 | (CH₂)₅CH₃ | (CH₂)₅CH₃ |
| 32 | CH(CH₃)₂ | CH(CH₃)₂ |

Exemplary embodiments include compounds having the formula (V) or a pharmaceutically acceptable salt form thereof:

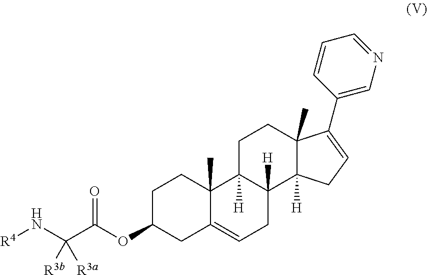

wherein non-limiting examples of R^{3a}, R^{3b}, and R⁴ are defined herein below in Table 4.

TABLE 4

| Example | R^{3a} | R^{3b} | R⁴ |
|---|---|---|---|
| 33 | H | H | H |
| 34 | H | CH₃ | H |
| 35 | CH₃ | H | H |
| 36 | H | CH(CH₃)₂ | H |
| 37 | CH(CH₃)₂ | H | H |
| 38 | H | CH₂CH(CH₃)₂ | H |
| 39 | CH₂CH(CH₃)₂ | H | H |
| 40 | H | 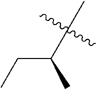 [SMILES: *[C@@H](C)CC] | H |
| 41 | 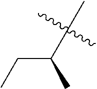 [SMILES: *[C@@H](C)CC] | H | H |
| 42 | H | CH₂CH₂SCH₃ | H |
| 43 | CH₂CH₂SCH₃ | H | H |
| 44 | H | CH₂Ph | H |
| 45 | CH₂Ph | H | H |
| 46 | H | 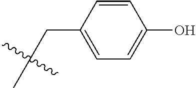 [SMILES: *Cc1ccc(O)cc1] | H |
| 47 | 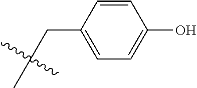 [SMILES: *Cc1ccc(O)cc1] | H | H |
| 48 | H | 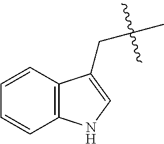 [SMILES: *Cc1c[nH]c2ccccc12] | H |
| 49 | 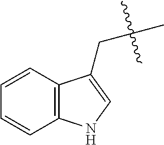 [SMILES: *Cc1c[nH]c2ccccc12] | H | H |
| 50 | H | CH₂OH | H |
| 51 | CH₂OH | H | H |
| 52 | H | 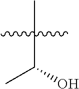 [SMILES: *[C@@H](C)O] | H |
| 53 | 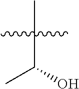 [SMILES: *[C@@H](C)O] | H | H |
| 54 | H | CH₂CONH₂ | H |
| 55 | CH₂CONH₂ | H | H |
| 56 | H | CH₂CH₂COCNH₂ | H |
| 57 | CH₂CH₂COCNH₂ | H | H |
| 58 | H | CH₂CH₂CO₂H | H |
| 59 | CH₂CH₂CO₂H | H | H |
| 60 | H | CH₂CO₂H | H |
| 61 | CH₂CO₂H | H | H |
| 62 | H | CH₂SH | H |
| 63 | CH₂SH | H | H |
| 64 | H | 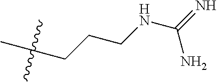 [SMILES: *CCCNC(=N)N] | H |
| 65 | 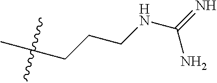 [SMILES: *CCCNC(=N)N] | H | H |
| 66 | H | CH₂CH₂CH₂CH₂NH₂ | H |
| 67 | CH₂CH₂CH₂CH₂NH₂ | H | H |
| 68 | H | 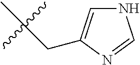 [SMILES: *Cc1c[nH]cn1] | H |
| 69 | 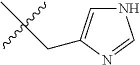 [SMILES: *Cc1c[nH]cn1] | H | H |

For the purposes of demonstrating the manner in which the compounds of embodiments herein are named and referred to herein, the compound having the formula:

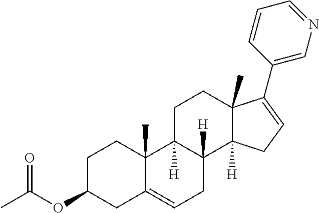

has the chemical name (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate. In some embodiments, it may be referred to as abiraterone acetate.

For the purposes of demonstrating the manner in which the compounds of embodiments herein are named and referred to herein, the compound having the formula:

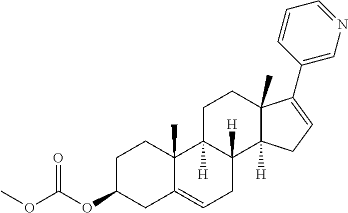

has the chemical name (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate.

For the purposes of demonstrating the manner in which the compounds of embodiments herein are named and referred to herein, the compound having the formula:

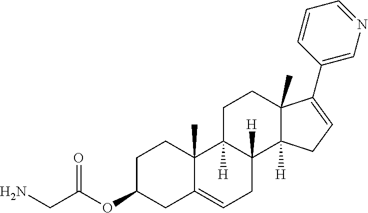

has the chemical name (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminoacetate.

In all of the embodiments provided herein, examples of suitable optional substituents are not intended to limit the scope of the claimed invention. The compounds of embodiments herein may contain any of the substituents, or combinations of substituents, provided herein.

Some embodiments described herein further relate to a process for preparing the cortisol lowering agents of embodiments herein.

Compounds of the present disclosure may be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations may be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions may also be used unless otherwise stated. Optimum reaction conditions may vary with the particular reactants or solvent used, but such conditions may be determined by one skilled in the art by routine optimization procedures. Those skilled in the art of organic synthesis will recognize that the nature and order of the synthetic steps presented may be varied for the purpose of optimizing the formation of the compounds described herein.

The processes described herein may be monitored according to any suitable method known in the art. For example, product formation may be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

In some embodiments, preparation of the compounds may involve protection and deprotection of various chemical groups. The need for protection and deprotection and the selection of appropriate protecting groups may be readily determined by one skilled in the art. The chemistry of protecting groups may be found, for example, in Greene et al., Protective Groups in Organic Synthesis, 2d. Ed. (Wiley & Sons, 1991), the entire disclosure of which is incorporated by reference herein for all purposes.

The reactions or the processes described herein may be carried out in suitable solvents which may be readily selected by one skilled in the art of organic synthesis. Suitable solvents typically are substantially nonreactive with the reactants, intermediates, and/or products at the temperatures at which the reactions are carried out, temperatures that may range from the solvent's freezing temperature to the solvent's boiling temperature. A given reaction may be carried out in one solvent or a mixture of more than one solvent. Depending on the particular reaction step, suitable solvents for a particular reaction step may be selected.

The compounds of embodiments described herein may be prepared by methods known in the art of organic chemistry. The reagents used in the preparation of the compounds of these disclosure may be either commercially obtained or may be prepared by standard procedures described in the literature. For example, compounds of embodiments described herein may be prepared according to the method illustrated in the reaction schemes below.

The reagents used in the preparation of the compounds of embodiments herein may be either commercially obtained or may be prepared by standard procedures described in the literature. Compounds in the genus may be produced by one of the following reaction schemes.

Compounds of formula (II) may be prepared according to the process outlined in Scheme 1.

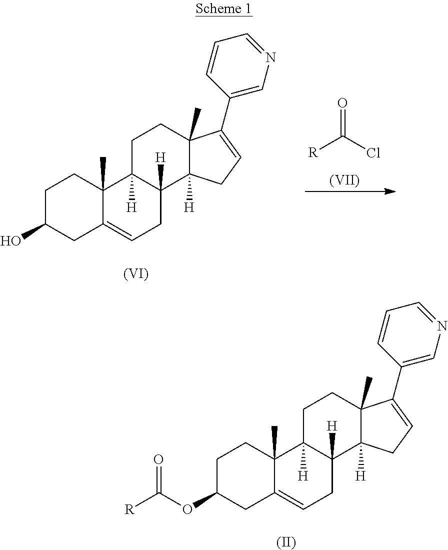

A suitably substituted compound of formula (VII), a known compound or compound prepared by known methods may be reacted with a compound of the formula (VI) in the presence of a bases such as, but not limited to, triethylamine, diisopropylethylamine, pyridine, 2,6-dimethylpyridine, N-methylmorpholine, and the like, in an organic solvent such as, but not limited to, methylene chloride, dichloroethane, tetrahydrofuran, 1,4-dioxane, N,N-dimethylformamide, and the like to provide a compound of the formula (II).

Alternatively, compounds of formula (II) may be prepared according to the process outlined in Scheme 2.

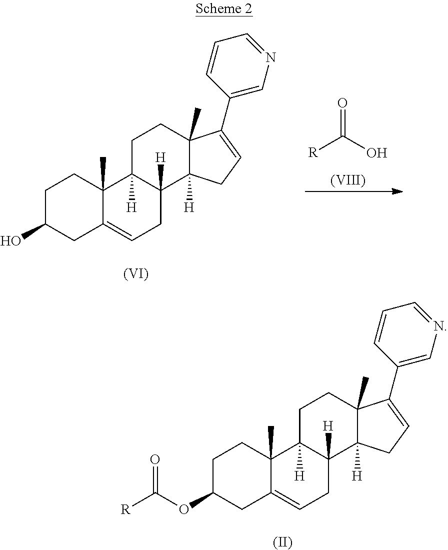

A suitably substituted compound of formula (VIII), a known compound or compound prepared by known methods may be reacted with a compound of the formula (VI) in the presence of a coupling agent such as, but not limited to, 1-ethyl-3-(3-dimethylaminopropyl) carbodiimide, N,N′-Dicyclohexylcarbodiimide, 2-(7-Aza-1H-benzotriazole-1-yl)-1,1,3,3-tetramethyluronium hexafluorophosphate and the like, in an organic solvent such as methylene chloride, tetrahydrofuran, 1,4-dioxane, dimethylforamide and the like, optionally in the presence of a base such as pyridine, triethyl amine, diisopropylethylamine and the like, to provide a compound of the formula (II).

Compounds of formula (V) may be prepared according to the process outlined in Scheme 3.

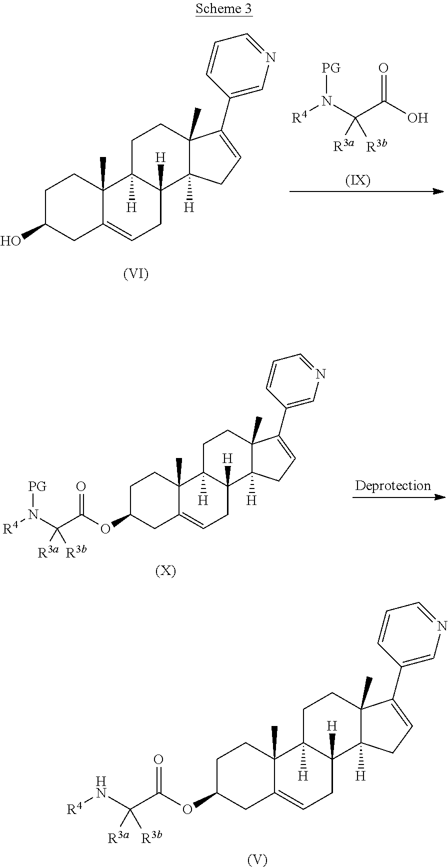

A suitably substituted compound of formula (IX), a known compound or compound prepared by known methods, wherein PG is a protecting group such as fluorenylmethyloxycarbonyl (Fmoc), carbobenzyloxy (Cbz), tert-butyloxycarbonyl (Boc) and the like, may be reacted with a compound of the formula (VI) in the presence of a coupling agent such as 1-ethyl-3-(3-dimethylaminopropyl) carbodiimide, N,N′-Dicyclohexylcarbodiimide, 2-(7-Aza-1H-benzotriazole-1-yl)-1,1,3,3-tetramethyluronium hexafluorophosphate and the like, in an organic solvent such as methylene chloride, tetrahydrofuran, 1,4-dioxane, dimethylforamide and the like, optionally in the presence of a base such as pyridine, triethylamine, diisopropylethylamine and the like, to provide a compound of the formula (X). The protecting group may be removed by treatment under suitable conditions such as 1) with acid, such as hydrogen chloride, trifluoroacetic acid, and the like in organic solvent such as methylene chloride, tetrahydrofuran, 1,4-dioxane, dimethylforamide, methanol, ethanol and the like, or 2) hydrogen in the presence of a catalyst such as palladium on activated carbon, platinum oxide and the like in an organic solvent such as ethyl acetate, methanol, ethanol tetrahydrofuran, 1,4-dioxane and the like or 3) with a base such as pyridine, triethylamine, diisopropylethylamine, piperidine and the like organic solvent such as methylene chloride, tetrahydrofuran, 1,4-dioxane, dimethylforamide, methanol, ethanol and the like to provide compounds of the formula (V).

Compounds of formula (III) may be prepared according to the process outlined in Scheme 4.

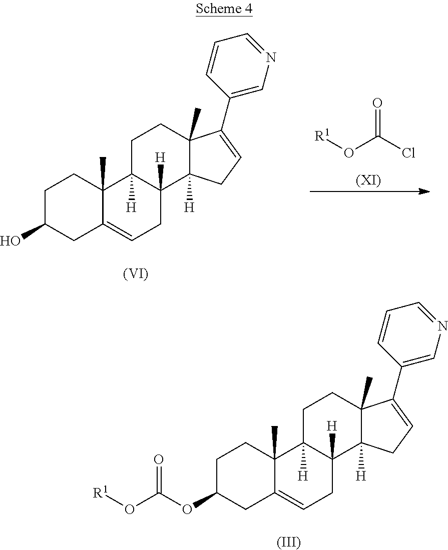

A suitably substituted compound of formula (XI), a known compound or compound prepared by known methods may be reacted with a compound of the formula (VI) in the presence of a bases such as, but not limited to, triethylamine, diisopropylethylamine, pyridine, 2,6-dimethylpyridine, N-methylmorpholine, and the like, in an organic solvent such as, but not limited to, methylene chloride, dichloroethane, tetrahydrofuran, 1,4-dioxane, N,N-dimethylformamide, and the like to provide a compound of the formula (III).

Compounds of formula (XII) may be prepared according to the process outlined in Scheme 5.

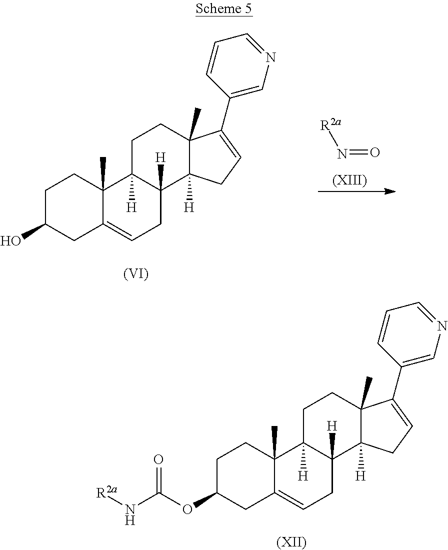

A suitably substituted compound of formula (XIII), a known compound or compound prepared by known methods may be reacted with a compound of the formula (VI) in an organic solvent such as, but not limited to methylene chloride, dichloroethane, tetrahydrofuran, 1,4-dioxane, N,N-dimethylformamide, and the like to provide a compound of the formula (XII).

Compounds of formula (IV) may be prepared according to the process outlined in Scheme 6.

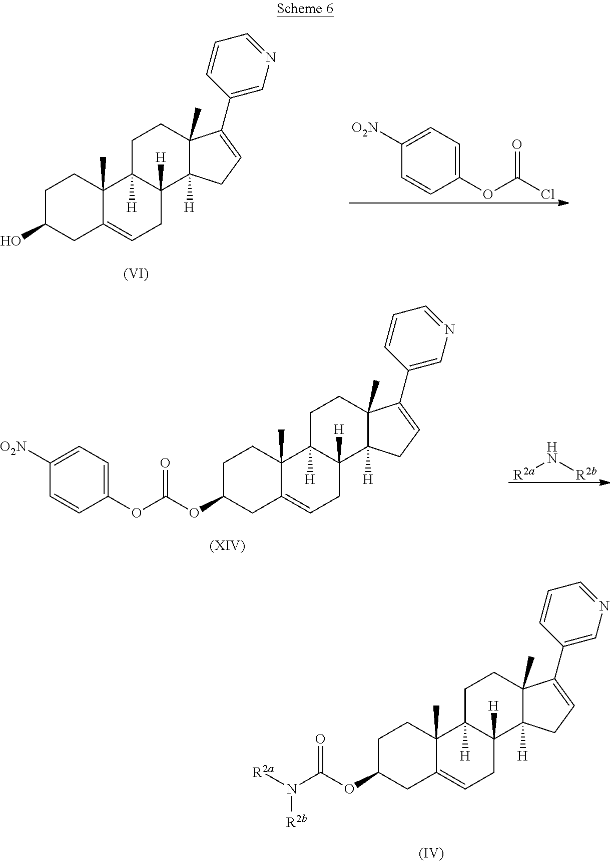

A suitably substituted compound of formula (VI), a known compound or compound prepared by known methods, may be reacted with a p-nitrophenylchloroformate in the presence of a bases such as, but not limited to, triethylamine, diisopropylethylamine, pyridine, 2,6-dimethylpyridine, N-methylmorpholine, and the like, in an organic solvent such as, but not limited to, methylene chloride, dichloroethane, tetrahydrofuran, 1,4-dioxane, N,N-dimethylformamide, and the like to provide a compound of the formula (XIV). A compound of the formula (XIV) may be then reacted with a compound of the formula (XV) a known compound or compound prepared by known methods, in an organic solvent such as, but not limited to, methylene chloride, dichloroethane, tetrahydrofuran, 1,4-dioxane, N,N-dimethylformamide, and the like, optionally with heating, optionally with microwave irradiation, to provide a compound of the formula (IV).

Some embodiments of the present disclosure also include compositions or formulations which comprise the cortisol lowering agents according to an embodiment herein. In general, the compositions of embodiments herein comprise an effective amount of one or more compounds of embodiments described herein and salts thereof according to an embodiment herein which are effective for providing cortisol lowering; and one or more excipients.

For the purposes of the present disclosure the term “excipient” and “carrier” are used interchangeably throughout the description and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be pan of a pH stabilizing system or coating to insure delivery of the ingredients safely to the stomach. The formulator may also take advantage of the fact the compounds of embodiments herein have improved cellular potency, pharmacokinetic properties, as well as improved oral bioavailability.

Some embodiments disclosed herein also provide pharmaceutical compositions that include at least one compound described in embodiments herein and one or more pharmaceutically acceptable carriers, excipients, or diluents. Examples of such carriers are well known to those skilled in the art and may be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients may also be incorporated into the pharmaceutical compositions.

Compounds of the present disclosure may be administered orally or parenterally, neat or in combination with conventional pharmaceutical carriers. Applicable solid carriers may include one or more substances which may also act as flavoring agents, lubrimayts, solubilizers, suspending agents, fillers, glidants, compression aids, binders or tablet-disintegrating agents, or encapsulating materials. In some embodiments, the compounds of embodiments herein may be formulated in conventional manner. Oral formulations containing a compound disclosed herein may comprise any conventionally used oral form, including tablets, capsules, buccal forms troches, lozenges and oral liquids, suspensions or solutions. In powders, the carrier may be a finely divided solid, which is an admixture with a finely divided compound. In tablets, a compound disclosed herein may be mixed with a carrier having the necessary compression properties in suitable proportions and compacted in the shape and size desired. The powders and tablets may contain up to 99% of the compound.

In some embodiments, capsules may contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

In some embodiments, useful tablet formulations may be made by conventional compression, wet granulation or dry granulation methods and utilize pharmaceutically acceptable diluents, binding agents, lubrimayts, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein may utilize standard delay or time-release formulations to alter the absorption of the compound(s). The oral formulation may also consist of administering a compound disclosed herein in water or fruit juice, containing appropriate solubilizers or emulsifiers as needed.

Liquid carriers may be used in preparing solutions, suspensions, emulsions, syrups, elixirs, and for inhaled delivery. A compound of the present disclosure may be dissolved or suspended in a pharmaceutically acceptable liquid carrier such as water, an organic solvent, or a mixture of both, or a pharmaceutically acceptable oils or fats. The liquid carrier may contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier may be an oily ester such as ethyl oleate and isopropyl myristate. Sterile liquid carriers may be used in sterile liquid form compositions for parenteral administration. The liquid carrier for pressurized compositions may be halogenated hydrocarbon or other pharmaceutically acceptable propellants.

Liquid pharmaceutical compositions, which are sterile solutions or suspensions, may be utilized by, for example, intramuscular, intraperitoneal or subcutaneous injection. Sterile solutions may also be administered intravenously. Compositions for oral administration may be in either liquid or solid form.

In some embodiments, pharmaceutical composition may be in unit dosage form, for example, as tablets, capsules, powders, solutions, suspensions, emulsions, granules, or suppositories. In such form, the pharmaceutical composition may be sub-divided in unit dose(s) containing appropriate quantities of the compound. The unit dosage forms may be packaged compositions, for example, pocketed powders, vials, ampoules, prefilled syringes or sachets containing liquids. Alternatively, the unit dosage form may be a capsule or tablet itself, or it may be the appropriate number of any such compositions in package form. Such unit dosage form may contain from about 1 mg/kg of compound to about 500 mg/kg of compound, and may be given in a single dose or in two or more doses. Such doses may be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it may be understood that an effective dosage may vary depending upon the particular compound utilized, the mode of administration, and severity of the condition being treated, as well as the various physical factors related to the individual being treated. In therapeutic applications, a compound of the present disclosure may be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some embodiments, it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present disclosure may be formulated into a liquid composition, a solid composition, or an aerosol composition. The liquid composition may include, by way of illustration, one or more compounds of the present disclosure dissolved, partially dissolved, or suspended in one or more pharmaceutically acceptable solvents and may be administered by, for example, a pump or a squeeze-actuated nebulized spray dispenser. The solvents may be, for example, isotonic saline or bacteriostatic water. The solid composition may be, by way of illustration, a powder preparation including one or more compounds of the present disclosure intermixed with lactose or other inert powders that are acceptable for intrabronchial use, and may be administered by, for example, an aerosol dispenser or a device that breaks or punctures a capsule encasing the solid composition and delivers the solid composition for inhalation. The aerosol composition may include, by way of illustration, one or more compounds of the present disclosure, propellants, surfactants, and co-solvents, and may be administered by, for example, a metered device. The propellants may be a chlorofluorocarbon (CFC), a hydrofluoroalkane (HFA), or other propellant that are physiologically and environmentally acceptable.

Compounds described herein may be administered parenterally or intraperitoneally. Solutions or suspensions of these compounds or a pharmaceutically acceptable salts, hydrates, or esters thereof may be prepared in water suitably mixed with a surfactant such as hydroxyl-propylcellulose. Dispersions may also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection may include sterile aqueous solutions or dispersions and sterile powders for the extemporaneous preparation of sterile injectable solutions or dispersions. In some embodiments, the form may sterile and its viscosity permits it to flow through a syringe. The them may be stable under the conditions of manufacture and storage and may be preserved against the contaminating action of microorganisms such as bacteria and fungi. The carrier may be a solvent or dispersion medium containing, for example, water, ethanol, polyol (e.g., glycerol, propylene glycol and liquid polyethylene glycol), suitable mixtures thereof, and vegetable oils.

Compounds described herein may be administered transdermally, i.e., administered across the surface of the body and the inner linings of bodily passages including epithelial and mucosal tissues. Such administration may be carried out using the compounds of the present disclosure including pharmaceutically acceptable salts, hydrates, or esters thereof, in lotions, creams, foams, patches, suspensions, solutions, and suppositories (rectal and vaginal).

Transdermal administration may be accomplished through the use of a transdermal patch containing a compound, such as a compound disclosed herein, and a carrier that may be inert to the compound, may be non-toxic to the skin, and may allow delivery of the compound for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the compound may also be suitable. A variety of occlusive devices may be used to release the compound into the blood stream, such as a semi-permeable membrane covering a reservoir containing the compound with or without a carrier, or a matrix containing the compound. Other occlusive devices are known in the literature.

Compounds described herein may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

Lipid formulations or nanocapsules may be used to introduce compounds of the present disclosure into host cells either in vitro or in vivo. Lipid formulations and nanocapsules may be prepared by methods known in the art.

The compounds of embodiments described herein may be administered in the conventional manner by any route where they are active. Administration may be systemic, topical, or oral. For example, administration may be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of embodiments described herein (either alone or in combination with other pharmaceuticals) may be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen may be to be adjusted or titrated by the clinician according to methods known to the clinician in order to obtain the optimal clinical response. The amount of compound to be administered may be that amount which is therapeutically effective. The dosage to be administered will depend on the characteristics of the subject being treated, e.g., the particular animal treated, age, weight, health, types of concurrent treatment, if any, and frequency of treatments, and may be easily determined by one of skill in the art (e.g., by the clinician).

Pharmaceutical formulations containing the compounds of embodiments described herein and a suitable carrier may be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of embodiments described herein. The active ingredients may be contained in such formulations with pharmaceutically acceptable diluents, fillers, disintegrants, binders, lubrimayts, surfactants, hydrophobic vehicles, water soluble vehicles, emulsifiers, buffers, humectants, moisturizers, solubilizers, preservatives and the like. The means and methods for administration are known in the art and an artisan may refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) may be consulted.

The compounds of embodiments described herein may be formulated for parenteral administration by injection, e.g., by bolus injection or continuous infusion. The compounds may be administered by continuous infusion subcutaneously over a period of about 15 minutes to about 24 hours. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

For oral administration, the compounds may be formulated readily by combining these compounds with pharmaceutically acceptable carriers well known in the art. Such carriers enable the compounds of embodiments described herein to be formulated as tablets, pills, drapes, capsules, liquids, gels, syrups, slurries, suspensions and the like, for oral ingestion by a patient to be treated. Pharmaceutical preparations for oral use may be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragamayth, methyl cellulose, hydroxypropylmethyl-cellulose, sodium carboxymethylcellulose, and polyvinylpyrrolidone (PVP). If desired, disintegrating agents may be added, such as, but not limited to, the cross-linked polyvinyl pyrrolidone, agar, or alginic acid or a salt thereof such as sodium alginate.

Dragee cores may be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which may be used orally include, but are not limited to, push-fit capsules made of gelatin, as well as soft, sealed capsules made of gelatin and a plasticizer, such as glycerol or sorbitol. The push-fit capsules may contain the active ingredients in admixture with filler such as, e.g., lactose, binders such as, e.g., starches, and/or lubrimayts such as, e.g., talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of, e.g., tablets or lozenges formulated in a conventional manner.

For administration by inhalation, the compounds for use according to an embodiment herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds of embodiments described herein may also be formulated in rectal compositions such as suppositories or retention enemas, e.g., containing conventional suppository bases such as cocoa butter or other glycerides.

In addition to the formulations described previously, the compounds of embodiments described herein may also be formulated as a depot preparation. Such long acting formulations may be administered by implantation (for example subcutaneously or intramuscularly) or by intramuscular injection.

Depot injections may be administered at about 1 to about 6 months or longer intervals. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of embodiments described herein, for example, may be applied to a plaster, or may be applied by transdermal, therapeutic systems that are consequently supplied to the organism.

Pharmaceutical compositions of the compounds also may comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of embodiments described herein may also be administered in combination with other active ingredients, such as, for example, adjuvants, protease inhibitors, or other compatible drugs or compounds where such combination may be seen to be desirable or advantageous in achieving the desired effects of the methods described herein. For example, the compounds of embodiments herein may be administered with ketoconazole, enantiomers thereof, mitotane (11 beta), pasireotide (somatostatin analogs), mifeptostone (cortisol receptor antagonists), cortisol synthesis receptors, carbamazepine, or analogs of each of the foregoing. In some embodiments, the compounds of embodiments herein may be administered with the 2S,4R enantiomer of ketoconazole.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pre gelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubrimayt component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

To increase the effectiveness of compounds of the present disclosure, it may be desirable to combine a compound with other agents effective in the treatment of the target disease. For example, other active compounds (i.e., other active ingredients or agents) effective in treating the target disease may be administered with compounds of the present disclosure. The other agents may be administered at the same time or at different times than the compounds disclosed herein.

Compounds of the present disclosure may be useful for the treatment or inhibition of a pathological condition or disorder in a mammal, for example, a human subject. Some embodiments of the present disclosure accordingly provide methods of treating or inhibition a pathological condition or disorder by providing to a mammal a compound of the present disclosure including its pharmaceutically acceptable salt) or a pharmaceutical composition that includes one or more compounds of the present disclosure in combination or association with pharmaceutically acceptable carriers. Compounds of the present disclosure may be administered alone or in combination with other therapeutically effective compounds or therapies for the treatment or inhibition of the pathological condition or disorder.

Some embodiments relate to a method for treating, delaying, slowing, or inhibiting the progression of diseases that involve overproduction of cortisol, including, for example, Cushing's Syndrome, obesity, headache, depression, hypertension, diabetes mellitus type II, metabolic syndrome, pseudo-Gushing syndrome, cognitive impairment, dementia, heart failure, renal failure, psoriasis, glaucoma, cardiovascular disease, stroke and incidentalomas, said method comprising administering to a subject in need thereof an effective amount of a compound according to an embodiment herein, wherein the disease that involves overproduction of cortisol is treated, delayed, slowed, or inhibited. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method for treating, delaying, slowing, or inhibiting the progression of diseases that involve overproduction of cortisol, including, for example, Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, wherein said method comprises administering to a subject a composition comprising an effective amount of one or more compounds according to an embodiment herein and an excipient. In some embodiments, the compound may include abiraterone or abiraterone acetate. In some embodiments, the compound may include a compound having formula (I) including hydrates, solvates, pharmaceutically acceptable salts, and complexes thereof in some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method for treating, delaying, slowing, or inhibiting the progression of diseases or conditions associated with Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas, and diseases that involve overproduction of cortisol, the method comprising administering to a subject an effective amount of a compound according to an embodiment herein. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method for treating, delaying, slowing, or inhibiting the progression of disease or conditions associated with Cushing's Syndrome, obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke and incidentalomas and diseases that involve overproduction of cortisol, wherein said method comprises administering to a subject a composition comprising an effective amount of one or more compounds according to an embodiment herein and an excipient. In some embodiments, the compound may include abiraterone or abiraterone acetate. In some embodiments, the compound may include a compound having formula (I) including, hydrates solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method for treating, delaying, slowing, or inhibiting the progression of disease or conditions associated with overproduction of cortisol. Said methods comprise administering, to a subject an effective amount of a compound or composition according to an embodiment herein. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method for treating, delaying, slowing, or inhibiting the progression of disease or conditions associated with overproduction of cortisol, wherein said method comprises administering to a subject a composition comprising an effective amount of one or more compounds according to an embodiment herein and an excipient. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof.

Some embodiments relate to a method of lowering the concentration of cortisol in the circulatory system, wherein the method comprises administering to a subject an effective amount of a compound or composition according to an embodiment herein. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof. In some embodiments, the subject has a higher than normal level of cortisol prior to treatment. In some embodiments, the subject has normal levels of cortisol prior to treatment.

Some embodiments relate to a method of lowering the concentration of cortisol in the circulatory system, wherein said method comprises administering to a subject a composition comprising an effective amount of one or more compounds according to an embodiment herein and an excipient. In some embodiments, the compound may include abiraterone or abiraterone acetate. In some embodiments, the compound may include a compound having formula (I) including hydrates, solvates, pharmaceutically acceptable salts, and complexes thereof. In some embodiments, the compound may include a compound having formula (II), including hydrates, solvates, pharmaceutically acceptable salts, and complexes thereof. In some embodiments, the compound may include a compound having formula (III), including hydrates, solvates, pharmaceutically acceptable salts, and complexes thereof. In some embodiments, the compound may include a compound having formula (IV), including hydrates, solvates, pharmaceutically acceptable salts, and complexes thereof. In some embodiments, the compound may include a compound having formula (V), including hydrates, solvates, pharmaceutically acceptable salts, and complexes thereof. In some embodiments, the subject has a higher than normal level of cortisol prior to treatment. In some embodiments, the subject has normal levels of cortisol prior to treatment.

Methods of embodiments herein may comprise administering a compound selected from (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propionate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyrate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentanoate; (3S,8R,9S,10R,13S,14S)-10,13-dimethyl-17-(pyridin-3-yl)-2,3,4,7,8,9,10,11,13,13,14,15-dodecahydro-1H-cyclopenta[a]phenanthren-3-yl hexanoate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl heptanoate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isobutyrate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pivalate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butyl carbonate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butylcarbamate; (3S,8R,9S,10R,13S,14S)-10,13-dimethyl-17-(pyridin-3-yl) 2,3,4,7,8,9,10,11,12,13,14,15-dodecahydro-1H-cyclopenta[a]phenanthren-3-yl pentylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dimethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diethylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipentylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dihexylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diisopropylcarbamate; (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl aminoacetate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl aminopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate; (2S,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate; (2R,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate; (2S,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate; (2R,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate; (R)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid; (S)-4-amino-5-(((3S,8R,9S,10R,13S,14S)-10,13-dimethyl-17-(pyridin-3-yl)-2,3,4,1,8,9,10,11,12,13,14,15-dodecahydro-1H-cyclopenta[a]phenanthren-3-yl)oxy)-5-oxopentanoic acid; (R)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid; (S)-3-amino-4-(((3S 8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate; (R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate; (S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate; or a pharmaceutically acceptable form thereof.

Non-limiting examples of compositions according to an embodiment herein include from about 0.001 mg to about 1000 mg of one or more compounds of the disclosure according to an embodiment herein and one or more excipients; from about 0.01 mg to about 100 mg of one or more compounds of the disclosure according to an embodiment herein and one or more excipients; from about 100 mg to about 250 mg of one or more compounds of the disclosure according to an embodiment herein and one or more excipients; from about 250 mg to about 500 mg of one or more compounds of the disclosure according to an embodiment herein and one or more excipients; from about 500 mg to about 750 mg of one or amore compounds of the disclosure according to an embodiment herein and one or more excipients; from about 750 mg to about 1000 mg of one or more compounds of the disclosure according to an embodiment herein and one or more excipients; and from about 0.1 mg to about 10 mg of one or more compounds of the disclosure according to an embodiment herein and one or more excipients.

In some embodiments, the compositions according to an embodiment herein are administered orally to a patient once daily. In some embodiments, the compositions according to an embodiment herein are administered orally to a patient twice daily. In some embodiments, the compositions according to an embodiment herein are administered orally to a patient three time per day. In some embodiments, the compositions according to an embodiment herein are administered orally to a patient once weekly.

In some embodiments, the compound may be administered at a dose of about 10 mg to about 2000 mg once daily. In some embodiments, the compound may be administered at a dose of about 100 mg to about 2000 mg once daily. In some embodiments, the compound may be administered at a dose of about 250 mg to about 2000 mg once daily. In some embodiments, the compound may be administered at a dose of about 250 mg to about 1000 mg once daily. In some embodiments, the compound may be administered at a dose of about 500 mg to about 2000 mg once daily. In some embodiments, the compound may be administered at a dose of about 500 mg to about 1000 mg once daily. In some embodiments, the compound is administered orally.

### Example 1

The following procedures may be utilized in evaluating and selecting compounds as cortisol lowering agents.

Adrenocorticotropic hormone (ACTH) Induced Cortisal Production Test: Male guinea pigs weighing 700 to 800 grams are randomized as per their body weight to create 3 groups of 6 animals (a vehicle group, a positive control group to be dosed with ACTH and vehicle, and a test group that will receive ACTH, vehicle and a compound of the disclosure). The animals are acclimated to their surroundings prior to initiation of the study. The animals in the test group and vehicle group are then dosed with vehicle or a vehicle containing the compound of the disclosure in 4.0 ml of the vehicle. After 30 minutes, the animals in the positive control group and test compound groups are injected with 20 IU ACTH intramuscularly. After 4 hours, blood is collected from the trunk of the animal, the plasma is separated, and plasma cortisol concentrations are assessed via HPLC/MS. Additional blood samples are collected from the trunk of the animal at 6, 8 and 12 hours, the plasma is separated, and plasma cortisol concentrations are assessed via. HPLC/MS.

### Example 2

TABLE 5

| | | Cyp17 | Cyp11 | Cyp21 | Cyp3A4 | Cyp2D6 | Cyp2C9 | | GPLM | HLM |
|---|---|---|---|---|---|---|---|---|---|---|
| Compound | MW | IC₆₀ (nM) | | | IC₆₀ (nM) | | | Solubility (uM) | t_{1/2} (min) | |
| abiraterone | 349 | 1.78 | 430 | 1550 | 969 | 2000 | 2000 | 1 | 7.7 | 60.0 |

Abiraterone acetate was screened for selectivity for Cyp17, Cyp21, Cyp11, Cyp3A4, Cyp2D6 and Cyp2C9. The in vitro enzyme profile shows abiraterone acetate to be highly selective for Cyp17.

### Example 3

A study was conducted to evaluate the effect of abiraterone acetate against the elevation of cortisol and testosterone after ACTH stimulation in male Guinea pigs. Each animal was dosed by oral gavage according to the following table at −0.5 hr, and then dosed by intramuscular injection with ACTH 25 IU/kg at 0 hr.

TABLE 6

| Treat- | | | Route | Dose | Dose | Dose |
|---|---|---|---|---|---|---|
| ment | | No. of | of | Level | Conc. | Volume |
| Group | Treatment | animals | admin. | (mg/kg) | (mg/mL) | (mL/kg) |
| 1 | vehicle | 4 | PO | 0 | 0 | 5 |
| 2 | arbiraterone | 4 | PO | 100 | 20 | 5 |

Animals were sedated under general inhalant anesthesia (3% isoflurane) and blood samples were retrieved at 1, 2 and 4 hrs via saphenous vein. Blood aliquots (400 uL) were collected in tubes coated with K2-EDTA, mixed gently, then kept on ice and centrifuged at 2,000 g for 5 minutes at 4° C. within 15 minutes of collection. The plasma was then harvested and kept frozen at −80° C. until further processing. Cortisol and testosterone levels were measured and are summarized in Table 7. As can be seen in Table 7, abiraterone significantly lower cortisol levels, as well as testosterone levels.

TABLE 7

| Cortisol, 25 IU/kg ACTH + vehicle | | | | | | testosterone, 25 IU/kg ACTH + vehicle | | | | | |
|---|---|---|---|---|---|---|---|---|---|---|---|
| Time | Guinea | Guinea | Guinea | Guinea | Mean | Time | Guinea | Guinea | Guinea | Guinea | Mean |
| (hr) | pig#1 | pig#2 | pig#3 | pig#4 | (ng/mL) | (hr) | pig#1 | pig#2 | pig#3 | pig#4 | (ng/mL) |
| 1 | 1530 | 1380 | 1600 | 919 | 1357 | 1 | 3.39 | 5.01 | 4.87 | 7.13 | 5.10 |
| 2 | 2150 | 1580 | 1540 | 1250 | 1630 | 2 | 9.35 | 5.15 | 6.08 | 7.83 | 7.10 |
| 4 | 2200 | 1820 | 1880 | 1430 | 1833 | 4 | 7.47 | 4.03 | 6.74 | 8.26 | 6.63 |
| Cortisol, 25 IU/kg ACTH + arbiraterone-100 mg/kg | | | | | | testosterone, 25 IU/kg ACTH + arbiraterone-100 mg/kg | | | | | |
| Time | Guinea | Guinea | Guinea | Guinea | Mean | Time | Guinea | Guinea | Guinea | Guinea | Mean |
| (hr) | pig#19 | pig#20 | pig#21 | pig#22 | (ng/mL) | (hr) | pig#19 | pig#20 | pig#21 | pig#22 | (ng/ml) |
| 1 | 692 | 821 | 659 | 970 | 786 | 1 | BQL | BQL | BQL | 0.501 | 0.501 |
| 2 | 574 | 720 | 470 | 876 | 660 | 2 | BQL | 0.473 | BQL | 0.520 | 0.497 |
| 4 | 320 | 461 | 325 | 529 | 409 | 4 | 0.328 | BQL | BQL | 0.461 | 0.395 |

1. A compound of formula (I), 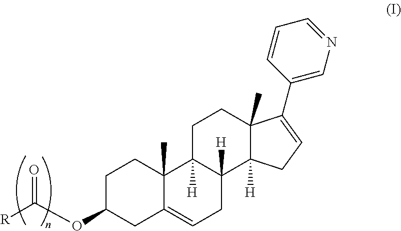
wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and 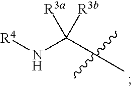
R¹ is selected from the group consisting of optionally substituted C₁-C₆ linear alkyl, optionally substituted C₁-C₆ branched allyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;
R⁴ is selected from a group consisting of hydrogen, optionally substituted C₁-C₆ linear alkyl, optionally substituted C₃-C₆ branched alkyl, optionally substituted benzyl, and optionally substituted heteroarylalkyl; and
n is 0 or 1,
hydrates, solvates, pharmaceutically acceptable salts, or complexes thereof,
wherein the compound does not comprise (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol, or (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate.

2. The compound of claim 1, wherein the compound is a compound of formula (II), 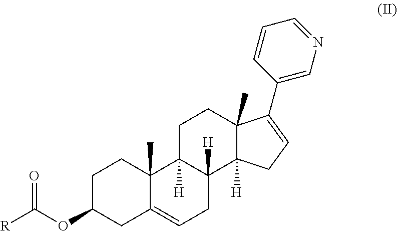
wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and 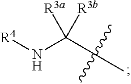
R¹ is selected from the group consisting of optionally substituted C₁-C₄ linear alkyl, optionally substituted C₁-C₆ branched alkyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl; and
R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl.

3. The compound of claim 1, wherein the compound is a compound of formula (III), 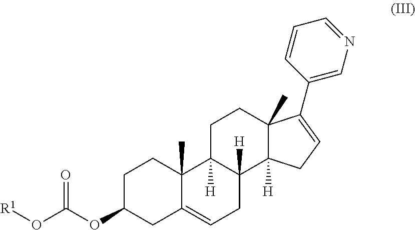
wherein R¹ is selected from the group consisting of optionally substituted C₁-C₆ linear alkyl, optionally substituted C₁-C₆ branched alkyl, and optionally substituted C₃-C₇ cycloalkyl.

4. The compound of claim 1, wherein the compound is a compound of formula (IV) 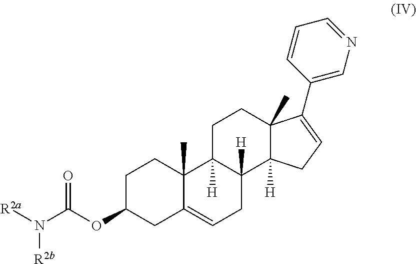
wherein R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl.

5. The compound of claim 1, wherein the compound is a compound of formula (V) 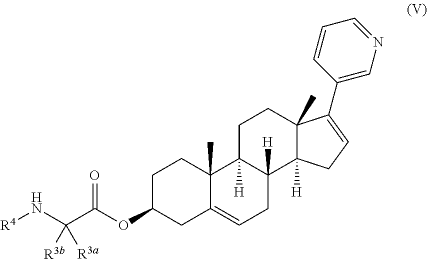
wherein R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl; and
R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl.

6. The compound of claim 1, wherein the compound is selected from the group consisting of
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propionate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyrate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl heptanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isobutyrate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pivalate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dimethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dihexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diisopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminoacetate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate;
(2S,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(2R,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(R)-(3S,8R,9S,10R,13S,14S)-10,43-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(2S,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(2R,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamine-5-oxopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate;
(R)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(S)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(R)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
and hydrates, solvates, pharmaceutically acceptable salts, or complexes thereof.

7. A pharmaceutical composition comprising an effective amount of a compound of the formula (I), 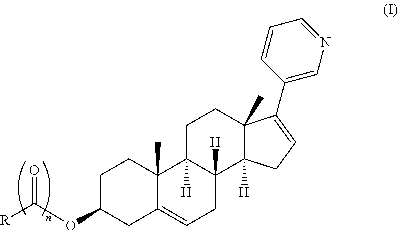
wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and 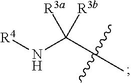
R¹ is selected from the group consisting of optionally substituted C₁-C₆ linear alkyl, optionally substituted C₁-C₆ branched alkyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;
R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl;
n is 0 or 1,
hydrates, solvates, pharmaceutically acceptable salts, or complexes thereof,
wherein the compound does not comprise (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol, or (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate; and
a pharmaceutically acceptable excipient.

8. A method for treating a disease associated with the overproduction of cortisol comprising administering to a subject in need thereof an effective amount of at least one compound of the formula (I), 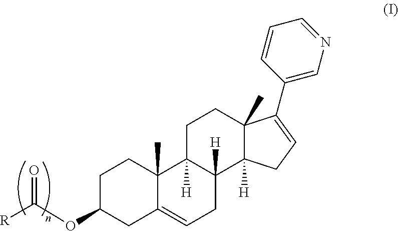
wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and 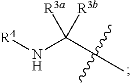
R¹ is selected from the group consisting of optionally substituted C₁-C₆ linear alkyl, optionally substituted C₁-C₆ branched alkyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{2b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;
R⁴ is selected from a group consisting of hydrogen, optionally substituted C₁-C₆ linear alkyl, optionally substituted C₁-C₆, branched alkyl, optionally substituted benzyl, and optionally substituted heteroarylalkyl; and
n is 0 or 1;
hydrates, solvates, pharmaceutically acceptable salts, or complexes thereof, and
wherein the disease associated with the overproduction of cortisol is treated.

9. The method of claim 8, wherein the at least one compound comprises a compound of formula (II), 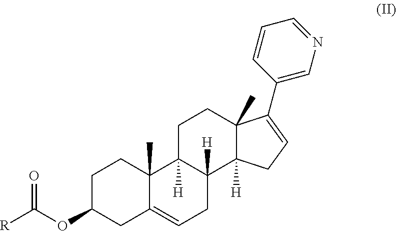
wherein R is selected from the group consisting of hydrogen, optionally substituted C₁-C₆ linear alkyl, optionally substituted C₁-C₅ branched alkyl, optionally substituted C₃-C₇ cycloalkyl, OR¹, NHR^{2a}, NR^{2a}R^{2b}, and 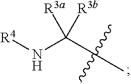
R¹ is selected from the group consisting of optionally substituted C₁-C₆ linear alkyl, optionally substituted C₁-C₄ branched alkyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{3b} are each independently selected from a group consisting of hydrogen, optionally substituted C₃-C₆ linear alkyl, optionally substituted C₁-C₆ branched alkyl, optionally substituted benzyl, and optionally substituted heteroarylalkyl; and
R⁴ is selected from a group consisting of hydrogen, optionally substituted C₃-C₆ linear alkyl, optionally substituted C₁-C₆ branched alkyl, optionally substituted benzyl, and optionally substituted heteroarylalkyl.

10. The method of claim 8, wherein the at least one compound comprises a compound of formula (III), 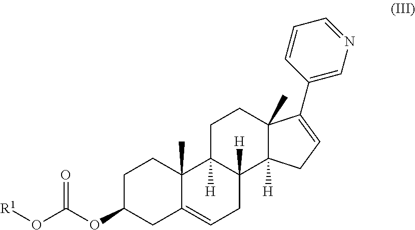
wherein R¹ is selected from the group consisting of optionally substituted C₁-C₆ linear alkyl, optionally substituted C₁-C₆ brandied alkyl, and optionally substituted C₃-C₇ cycloalkyl.

11. The method of claim 8, wherein the at least one compound comprises a compound of formula (IV) 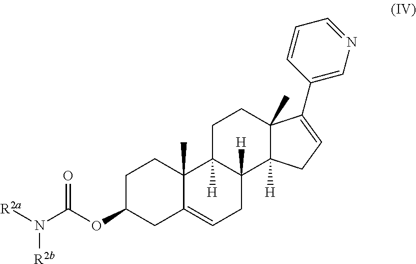
wherein R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl.

12. The method of claim 8, wherein the at least one compound comprises a compound of formula (V) 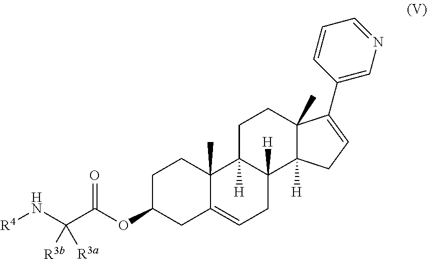
wherein R^{3a} and R^{3b} are each independently selected from group consisting of hydrogen, optionally substituted C₁-C₆ linear alkyl, optionally substituted C₁-C₆ branched alkyl, optionally substituted benzyl, and optionally substituted heteroarylalkyl; and
R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl.

13. The method of claim 8, wherein the at least one compound is selected from the group consisting of
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propionate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyrate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl heptanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isobutyrate;
(3S,8R,9S,10R,13S,14S)-10,13-dimethyl-11-(pyridin-3-yl)-2,3,4,7,8,9,10,11,12,13,14,15-dodecahydro-1H-cyclopenta[a]phenanthren-3-yl pivalate;
(3S,8R,9S,10R,3S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dimethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dihexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diisopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminoacetate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate,
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl amino-4-methylpentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl amino-4-methylpentanoate;
(2S,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(2R,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(2S,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(2R,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(R)-(3S,8R,9S,10R,13S,14S)-10,13-dimethyl-17-(pyridin-17-yl)-2,3,4,7,8,9,10,11,12,13,14,15-dodecahydro-1H-cyclopenta[a]phenanthren-3-yl 2,4-diamino-4-oxobutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate;
(R)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(S)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(R)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
or a pharmaceutically acceptable form thereof.

14. The method of claim 8, wherein the at least one compound is administered in a composition further comprising at least one excipient.

15. The method of claim 8, wherein the diseases associated with the overproduction of cortisol is Cushing's syndrome.

16. The method of 8, wherein the disease associated with the overproduction of cortisol is selected from obesity, headache, depression, hypertension, diabetes mellitus type II, metabolic syndrome, pseudo-Cushing syndrome, cognitive impairment, dementia, heart failure, renal failure, psoriasis, glaucoma, cardiovascular disease, stroke, incidentalomas, and combinations thereof.

17. The method of claim 8, wherein the at least one compound is (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol.

18. The method of claim 8, wherein the at least one compound is (3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate.

19. A method of lowering cortisol concentration in the circulatory system, the Method comprising administering to a subject in need thereof an effective amount of at least one compound of the formula (I), 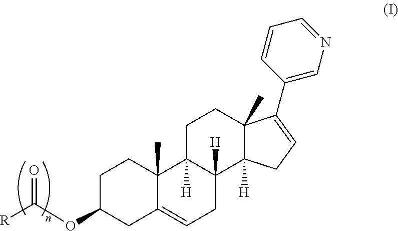
wherein R is selected from the group consisting of hydrogen, optionally substituted C₁-C₆ linear alkyl, optionally substituted C₁-C₆ branched alkyl, optionally substituted C₃-C₇ cycloalkyl, OR¹, NHR^{2a}, NR^{2a}R^{2b}, and 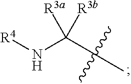
R¹ is selected from the group consisting of optionally substituted C₁-C₆ linear alkyl, optionally substituted C₁-C₆ branched alkyl, and optionally substituted C₃-C₇ cycloalkyl;
R^{2a} and R^{2b} are each independently selected from a group consisting of hydrogen, optionally substituted C₁-C₆ linear alkyl, optionally substituted C₁-C₆ branched alkyl, and optionally substituted C₃-C₇ cycloalkyl;
R^{3a} and R^{3b} are each independently selected from a group consisting of hydrogen, optionally substituted C₁-C₆ linear alkyl, optionally substituted C₁-C₆ branched alkyl, optionally substituted benzyl, and optionally substituted heteroarylalkyl;
R⁴ is selected from a group consisting of hydrogen, optionally substituted C₁-C₆ linear alkyl, optionally substituted C₁-C₆ branched alkyl, optionally substituted benzyl, and optionally substituted heteroarylalkyl; and
n is 0 or 1;
hydrates, solvates, pharmaceutically acceptable salts, or complexes thereof,
wherein the subject in need thereof has a higher than normal level of cortisol; and
wherein the cortisol concentration in the circulatory system is towered.

20. The method of claim 19, wherein the at least one compound is selected from the group consisting of
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-ol;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl acetate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propionate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyrate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl heptanoate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isobutyrate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pivalate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethyl carbonate;
(3S,8R,9S,10R,13S,14S)-10,13-dimethyl-17-(pyridin-3-yl)-2,3,4,7,8,9,11,11,12,13,14,15-dodecahydro-1H-cyclopenta[a]phenanthren-3-yl propyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butyl carbonate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl methylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl ethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl propylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl pentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl hexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl isopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl tert-butylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dimethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diethylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dibutylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dipentylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl dihexylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl diisopropylcarbamate;
(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminoacetate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-aminopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylbutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-methylpentanoate;
(2S,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(2R,3S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-methylpentanoate;
(R)-(3 S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-4-(methylthio)butanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-phenylpropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(4-hydroxyphenyl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-indol-3-yl)propanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(S)-(3S,8R,9S,10R,13S,14S)-18,13-dimethyl-17-(pyridin-3-yl)-2,3,4,7,8,9,10,11,12,13,14,15-dodecahydro-1H-cyclopenta[a]phenanthren-3-yl 2-amino-3-hydroxypropanoate;
(2S,3R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(2R,3R)-(3S,8R,9S,10R,13 S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-hydroxybutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,4-diamino-4-oxobutanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,5-diamino-5-oxopentanoate;
(R)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(S)-4-amino-5-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-5-oxopentanoic acid;
(R)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-3-amino-4-(((3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl)oxy)-4-oxobutanoic acid;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-mercaptopropanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-guanidinopentanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-5-guanidinopentanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2,6-diaminohexanoate;
(R)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
(S)-(3S,8R,9S,10R,13S,14S)-10,13-dimethyl-17-(pyridin-3-yl)-2,3,4,7,8,9,10,11,12,13,14,15-dodecahydro-1H-cyclopenta[a]phenanthren-3-yl 2-amino-3-(1H-imidazol-4-yl)propanoate;
or a pharmaceutically acceptable form thereof.
